(19) United States
(12) Patent Application Publication
(10) Pub. No.: US 2004/0102818 A1
(43) Pub. Date: May 27, 2004

(54) Method and system for controlling blood pressure

(76) Inventors: Said I. Hakky, Largo, FL (US); A-Hamid Hakki, Dunedin, FL (US)

Correspondence Address: ROSENBERG, KLEIN & LEE, 3458 ELLICOTT CENTER DRIVE-SUITE 101, ELLICOTT CITY, MD, 21043, US

(21) Appl. No.: 10/303,970

(22) Filed: Nov. 26, 2002

(51) Int. Cl. A61N 1/18

(52) U.S. Cl. 607/44

(57) ABSTRACT

A system and method for controlling blood pressure are provided. The system (10) includes an energy dispensing means (14) which is secured to a user's epidermis and positioned adjacent a blood vessel (22) of the user. The energy dispensing means (14) is in electrical communication with a control means (18). Through the control means (18), a user may control pulse rate, pulse amplitude, pulse width, and duration of treatment. Through the delivery of a series of energetic pulses to the blood vessel (22), the user's blood pressure will be lowered.

### BACKGROUND OF THE INVENTION

1. Field of the Invention

The subject invention relates to a system and method for controlling blood pressure. In particular, the present invention directs itself to an energy dispensing means which is fixed to a user's epidermis. More particularly, the energy dispensing means is in electrical communication with a control means. The control means allows a user to control pulse width, pulse duration, and pulse rate of a series of energy pulses produced by the energy dispensing means and transmitted through a blood vessel of the user.

Further, the invention directs itself to the monitoring of the user's blood pressure subsequent to application of the energy pulses to the user's blood vessel. Blood vessel treatment with a series of energetic pulses will lower the user's blood pressure.

Still further, the invention relates to a non-invasive blood pressure monitor and control system for both monitoring the user's blood pressure and controlling the patient's blood pressure without any invasive techniques. In particular, control is obtained through a control mechanism electrically coupled to electrodes which are placed contiguous and external the epidermis of the patient. Monitoring of the patient's blood is obtained through use of standard techniques including a cuff mounted to the epidermis of the patient and coupled to a standard well-known pressure monitoring system. In this manner, both control and monitoring of the blood pressure of the patient is obtained in a non-invasive procedure.

2. Prior Art

Systems and methods for the treatment of hypertension and high blood pressure are well-known in the art. Generally, treatments for hypertension are directed towards special diets, exercise, and weight loss. Additionally, blood pressure may be controlled through oral medications or through nerve stimulation. In general, however, the oral medications for hypertension are expensive and can have many side effects. Additionally, systems which control hypertension disorder through electrical stimulation of nerve cells can cause neural damage. Direct stimulation of the blood vessel, however, offers a minimum of risk to the patient.

One such prior art method is shown in U.S. Pat. No. 6,178,352. This reference is directed to a method of blood pressure moderation. The method teaches the control of blood pressure in a patient with high blood pressure or low blood pressure utilizing a non-invasive nerve stimulation device applied to the wrist. This system, however, utilizes stimulation only of the nerves and not the blood vessels.

Another such prior art treatment method is shown in U.S. Pat. No. 5,707,400. This method is directed to a treatment of refractory hypertension by nerve stimulation. This system uses a stimulating signal applied to the nerve and includes a method for programming various signal parameters. However, the stimulation takes place directly on the nerve cells and is not directed towards stimulation of a blood vessel.

None of the prior art provides for a method of treatment wherein a series of energetic pulses are transmitted to and through a blood vessel. The application of energy pulses to the blood vessel in order to control hypertension disorder is a non-invasive means of treatment with very few side effects for the patient.

The application of energy pulses to the blood vessel of the patient in order to control hypertension disorder as provided in the present invention is not seen in the prior art when taken with respect to non-invasive systems. The non-invasive nature of the subject Patent Application minimizes any possible side effects to the patient and optimizes the response time for control of the blood pressure.

### SUMMARY OF THE INVENTION

The present invention provides for a system and method for controlling a user's blood pressure. The system includes an energy dispensing means which is fixed to a user's epidermis adjacent a user's blood vessel. The energy dispensing means is in electrical communication with a control means. Via the control means, the user may control the pulse rate, pulse width, and pulse duration of the energy pulse generated by the energy dispensing means. The energy pulse may be in the form of electrical energy, electromagnetic energy, laser or ultrasonic waves. Through application of a series of energetic pulses through the blood vessel, the user's blood pressure will be lowered.

It is a principle objective of the subject system and method for controlling a user's blood pressure to provide an energy dispensing means fixed to a user's epidermis adjacent to a blood vessel.

It is a further objective of the subject system and method for controlling blood pressure to provide a control means in electrical communication with the energy dispensing means.

It is a further objective of the subject invention to provide user control over pulse width, pulse rate, and pulse duration of the series of energetic pulses produced by the energy dispensing means.

It is an important objective of the present invention to provide a means for monitoring a user's blood pressure subsequent to the application of energetic pulses to the user's blood vessel.

It is a very important object of the present invention to provide a non-invasive mechanism for monitoring a user's blood pressure subsequent to the application of energetic pulses to the user's blood vessel which has the effect of providing external stimulation and maintaining a non-invasive technique system for optimizing control and minimizing any possible side effects.

### BRIEF DESCRIPTION OF THE DRAWINGS

FIG. 1 is a perspective view of the subject system for controlling and monitoring blood pressure applied to a user's arm.

### DESCRIPTION OF THE PREFERRED EMBODIMENTS

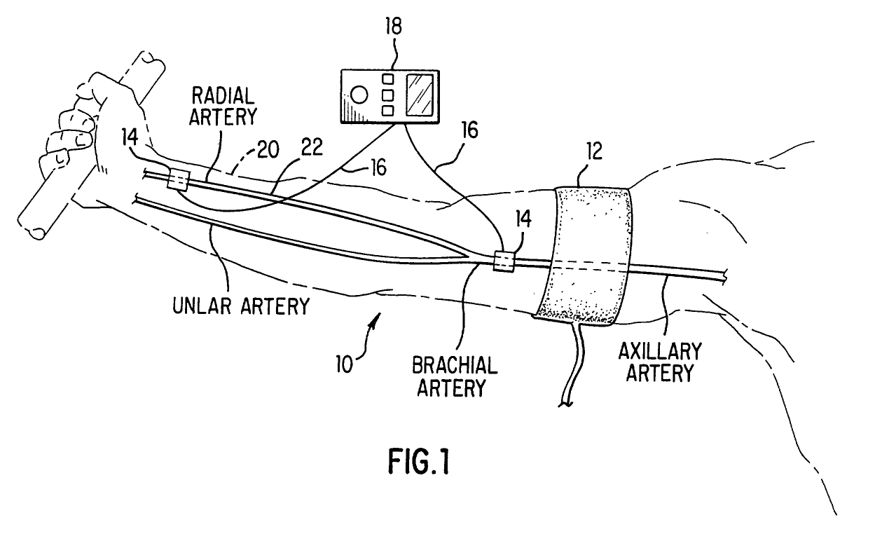

Referring now to FIG. 1, there is shown a system for controlling the blood pressure of a user. The system 10 includes energy dispensing means 14, control means 18, and electrical leads 16. The system 10 is a completely non-invasive system which is secured and mounted external to the epidermis of the patient and may be set up with a minimization of complexity and discomfort to the patient. This non-invasive technique permits the optimization of the control and monitoring of the blood pressure of a patient while minimizing any possible side effects which may occur.

The energy dispensing means 14 is secured to the user's epidermis adjacent a blood vessel 22. In FIG. 1, blood vessel 22 is illustrated as being in the arm 20 of the user. However, the energy dispensing means 14 may be applied to the epidermis of the user at any appropriate location on the body.

The energy-dispensing means illustrated in FIG. 1 is shown as being a pair of electrodes 14. The energy dispensing means, however, may produce pulses of electrical energy, pulses of electromagnetic energy, or pulses of ultrasonic energy. In the example shown in FIG. 1, electrodes 14 produce a series of electrical pulses.

The electrodes 14 may be any standard adhesively applied electrodes designed for application to the epidermis. One such set of electrodes is the Red Dot Repositionable Electrode produced by 3M Corp., of St. Paul, Minn.

The energy dispensing means 14 are in electrical communication with control means 18 via power leads 16. Control means 18 allows the user to control the pulse width, pulse rate, and duration of treatment. Electrical stimulators for medical treatment are well-known in the art. One such stimulator/control device is the Stimtech PT1 Stimulator, produced by Stimtech Products of Southbridge, Mass.

Human blood vessels, such as blood vessel 22 illustrated in FIG. 1, have an epithelial lining called the endothelium. Surrounding the endothelium is a smooth muscle layer that controls the diameter of the blood vessel. In a patient suffering from hypertension, the diameter of the blood vessels is usually in a contracted state. In the case of a patient with low blood pressure, these blood vessels are in a state of relaxation. The blood vessels may be arteries, capillaries, or veins.

The endothelium has a complex structure which produces chemical agents that can relax the smooth muscle of the blood vessels. Prostacyclin is formed from arachidonic acid through the action of cyclooxygenase and prostacyclin synthethase in the endothelium, which elicits relaxation of the smooth muscles of the artery through increases in the level of cyclic adenosine monophosphate. Stimulation of the endothelial cells with acetyl choline results in the formation and release of endothelium-derived relaxing factor (EDRF), which is nitric oxide (NO). The EDRF stimulates guanylyl cyclase to increase cyclic guanosine monophosphate in the vascular smooth muscle to produce relaxation. These chemo-receptors control the prolonged changes in the blood pressure.

The blood vessels are surrounded by adrenergic nerve fibers. These adrenergic nerve fibers can cause constriction of the blood vessels if stimulated. The blood vessels are also surrounded by cholinergic nerve fibers. These cholinergic nerves can cause vasodilation if stimulated. The nerves surrounding the medium size blood vessels are also connected to the vasoconstriction and vasodilation centers located in the reticular substance of the medulla and the lower third of the pons in the brain.

Under normal condition, the vasoconstrictor area of the vasomotor center transmits signals continuously to the vasoconstrictor nerve fibers. These signals are fired at one and a half impulses per second. This is called the sympathetic tone. The nervous system controls the rapid changes in the blood pressure.

Through stimulation of the blood vessels with a series of energy pulses, the blood pressure of the user is found to drop. One possible reason for the drop in blood pressure could be the release of nitric acid from the cells of the intima. The drop in blood pressure could also be due to antidromic nerve impulses set by the energetic stimulation. The energetic stimulation may lead to setting up wide range vasodilation, thus a drop in the blood pressure. The blood pressure drop could further be due to stimulation of the vasodilator center in the brain; i.e., a central effect. Stimulation of the carotid body at the bifurcation of the carotid artery will lead to reflex drop in blood pressure. This action is central. The blood pressure drop could additionally be due to the stimulation of the cholinergic sympathetic nerves surrounding the blood peripheral blood vessels; i.e., peripheral action.

FIG. 1 illustrates a blood pressure cuff 12 applied to the arm 20 of the user. Subsequent to application of the series of energetic pulses, it may be useful to measure the blood pressure of the user for purposes of comparison.

In tests of the system and method, a white male, age 57, with mild hypertension was subjected to electrical stimulation. Three different modes were tested: first, in a normal mode, the pulse width used was 60 microseconds at a rate of 80 pulses per second, and with a pulse amplitude of 2-6 mA. The duration of treatment was 2-3 minutes. Next, in a burst mode, the pulse width used was 200 microseconds at a rate of 150 pulses per second. The pulse amplitude was 2-6 mA and the treatment lasted for a duration of 2-3 minutes. Lastly, in a modulation mode, the pulse width tested was 60 microseconds at a pulse rate of 50 pulses per second. The amplitude was 2-6 mA and the duration was 2-3 minutes.

Two skin electrodes were applied to the patient. One skin electrode was applied to the superficial radial artery at the wrist. The other skin electrode was applied to the brachial artery at the antecubital fossa of the forearm.

The blood pressure of the patient before the application of the electric current ranged between 130-135 mm of mercury systolic and 85-90 mm of mercury diastolic. The blood pressure was measured using an Omron blood pressure HEM-704C monitor. The volunteer was not on any blood pressure medication. The blood pressure was taken several times before and after the application of the electrical stimulation. The experiment was repeated over several weeks to confirm the findings.

Within a few minutes of the application of the electric current, the systolic blood pressure dropped to the range of 109-117 mm of mercury and the diastolic blood pressure dropped to the range of 69-75 mm of mercury. The burst mode generated a more dramatic drop in the patient's blood pressure. However, there was a measurable, significant drop in the blood pressure using all three modes.

The blood pressure was at a low level for 10-60 minutes after the end of the electrical stimulation. Following numerous trials, the blood pressure stayed low for 3-6 hours. The blood pressure, however, was found to go back to the original range of 130-135 mm systolic and 85-88 mm diastolic during exercise, strenuous work, or tension. Upon subsequent electrical stimulation, the blood pressure dropped again within one minute of the electrical application.

The blood pressure cuff was tried on both the arm where the electrical leads were placed, and also on the patient's opposing arm. No difference was observed in the response to the lowering of the blood pressure by electrical stimulation. The leads were also interchanged and no effect was noticed.

In addition to electrical stimulation, energy dispensing means 14 may also take the form of ultrasonic transducers for creating ultrasonic energy waves in the patient's blood pressure, or a means for generating electromagnetic energy.

Although-this invention has been described in connection with specific forms and embodiments thereof, it will be appreciated that various modifications other than those discussed above may be resorted to without departing from the spirit or scope of the invention. For example, functionally equivalent elements may be substituted for those specifically shown and described, proportional quantities of the elements shown and described may be varied, and in the method steps described, particular steps may be reversed or interposed, all without departing from the spirit or scope of the invention as defined in the appended

What is claimed is:

1. A method for controlling blood pressure comprising the steps of:
(a) securing an energy dispensing means on a user's epidermis, said energy dispensing means being located contiguous said user's epidermis and adjacent a blood vessel; and,
(b) delivering a series of energy pulses to said blood vessel from said energy dispensing means.

2. The method for controlling blood pressure as recited in claim 1, wherein said energy dispensing means comprises a pair of electrodes.

3. The method for controlling blood pressure as recited in claim 1, wherein said energy dispensing means comprises at least one ultrasonic transducer.

4. The method for controlling blood pressure as recited in claim 1, wherein said energy pulses are electromagnetic pulses.

5. The method for controlling blood pressure as recited in claim 1, wherein said step of delivering a series of energetic pulses is followed by monitoring of said user's blood pressure.

6. The method for controlling blood pressure as recited in claim 2, wherein said energy pulses have a pulse width of 60 microseconds.

7. The method for controlling blood pressure as recited in claim 2, wherein said energy pulses have a pulse width of 200 microseconds.

8. The method for controlling blood pressure as recited in claim 2 wherein said energy pulses have a pulse rate of 80 pulses per second.

9. The method for controlling blood pressure as recited in claim 2 wherein said energy pulses have a pulse rate of 150 pulses per second.

10. The method for controlling blood pressure as recited in claim 2 wherein said energy pulses have a pulse rate of 50 pulses per second.

11. The method for controlling blood pressure as recited in claim 2 wherein said energy pulses have an amplitude in the range of 2-6 mA.

12. The method for controlling blood pressure as recited in claim 2, wherein duration of treatment is in the range of 2-3 minutes.

13. A system for controlling blood pressure comprising:
an energy dispensing means for delivering a series of energy pulses to a blood vessel of a user, said energy dispensing means being secured to said user's epidermis and being located contiguous said user's epidermis and adjacent said blood vessel; and,
control means for controlling said energy dispensing means, said control means being in electrical communication with said energy dispensing means.

14. The system for controlling blood pressure as recited in claim 1, wherein said energy dispensing means comprises a pair of electrodes.

15. The system for controlling blood pressure as recited in claim 13, wherein said energy dispensing means comprises at least one ultrasonic transducer.

16. The system for controlling blood pressure as recited in claim 13, wherein said energy pulses are electromagnetic pulses.

17. The system for controlling blood pressure as recited in claim 13, wherein said control means controls pulse width of said energy pulses.

18. The system for controlling blood pressure as recited in claim 13, wherein said control means controls pulse rate of said energy pulses.

19. The system for controlling blood pressure as recited in claim 13, wherein said control means controls duration of treatment.

20. The system for controlling blood pressure further comprising a blood pressure monitor applied to said user for monitoring said user's blood pressure subsequent to actuation of said energy dispensing means.
